(19) United States
(12) Patent Application Publication
(10) Pub. No.: US 2007/0276438 A1
(43) Pub. Date: Nov. 29, 2007

(54) BACK ALIGNMENT DEVICE

(76) Inventors: Michelle Meglin, Wilmington, NC (US); Michael Meglin, Wilmington, NC (US); Michael Lovette, Goleta, CA (US); Allen Meglin, Savannah, GA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/420,563

(22) Filed: May 26, 2006

(51) Int. Cl. A61F 5/00

(52) U.S. Cl. 606/237

(57) ABSTRACT

A back aligner device includes an elastic body of material having on one surface thereof a shape approximately complementary to posterior surfaces of a plurality of human vertebrae in a desired alignment, and having an another surface thereof a mounting structure capable of holding the article in a desired position. A kit of back aligners, from which one with a suitable profile is selected is also provided. A method aligning plural vertebrae in a human having need thereof, comprises: providing an elastic support having a surface shape complementary to a plurality of aligned vertebrae; relaxing spinal muscles adjacent the plural vertebrae to be aligned; and pressing the spine against the elastic support so as to align the spine.

### BACKGROUND OF INVENTION

Aspects of embodiments of the present invention relate generally to methods and apparatus useful for improving the alignment of vertebrae in the spine of a human in need of improved spinal alignment. More particularly, aspects of embodiments of the invention relate to methods and apparatus particularly suitable for adaptation to self-treatment.

FIGS. 1 and 2 show portions of a human thoracic spine which help illustrate the context in which the invention can be used. Although the thoracic spine is used for illustrative purposes, as will be seen, the concepts of the invention apply to the cervical spine and lumbar spine, as well.

The thoracic spine includes plural vertebrae 100 connected to each other and to ribs 102 through various joints. Three such joints that can become misaligned are the costovertebral joint 101, at which the head 108 of a rib 102 joins a vertebra 100 at the vertebral body 103, the costotransverse process joint 104, at which the neck 105 of the rib 102 joins a vertebra 100 at a transverse process 106, and the facet joints 107, at which each vertebra 100 joins the vertebra 100 above or below.

The spine and surrounding structures are heavily innervated, i.e., numerous nerves pass through the region. The spinal cord passes through each vertebra via a vertebral canal 109, and bundles of nerves branch out through passageways called neuroforamen 110. If any of the spinal joints become distracted, compressed or twisted, the result is often painful or severely uncomfortable because the nerves passing through the neuroforamen 110 become compressed, pinched or otherwise disturbed. Moreover, the joints themselves are innervated and improper alignment may cause discomfort due to compression, extension or other activation of the nerves in the joints due to improper joint position. Restoring alignment to the vertebrae 100 and their joints 101, 104, 107, can improve comfort substantially. Many people employ stretching exercises or visit chiropractors to correct the alignment of their vertebrae. This correction is frequently accompanied by a noticeable “crack” sound, believed to be due the rapid expansion of gas accompanying the movement of fluid into or out of the joint space.

The result of realignment, aside from the characteristic “crack” sound is a substantial reduction in pain or improvement in comfort, as the neuroforamen and other structures and passageways assume their natural relationships, with adequate space for the passage of affected nerve bundles.

Until now, self-treatment of the minor misalignments susceptible to such self-treatment has been relatively difficult and risky. A number of apparatus and methods have been developed to improve self-treatment of minor spinal misalignments, while incurring minimal risk. Each has various disadvantages. Some of the apparatus include:
adjustable mattresses;
pillows for correcting alignment;
a board to lie upon with two ridges for aligning spinous processes;
inversion beds for harnessing gravity to improve alignment;
chiropractic manipulators;
massage chairs; and
neck traction devices.

Mattresses, pillows, massage chairs and the like may offer temporary relief while actively used, but the benefit is often lost as soon as the individual gets up off the mattress, pillow, massage chair or the like because no long-lasting adjustment has been made. Chiropractic manipulation and neck traction devices may offer longer-lasting relief because they make longer-lasting adjustments to the alignment of the spine, but they present significant risk to the individual because the manipulations produced are of a nature that can easily cause permanent damage to nerves and joints. Chiropractic manipulation relies on relatively high-velocity movements that can go out of control and cause irreparable damage, while neck traction stretches the spine in the proper direction, but does not use a structure that assures proper alignment will result. If the muscles and tendons of the back are laterally imbalanced, neck traction is thus unlikely to produce the desired result.

### SUMMARY OF INVENTION

According to various aspects of embodiments of the invention, improved methods and apparatus are provided for self-treating minor misalignment of vertebrae in the spine of a human in need of improved spinal alignment.

An article according to some aspects of embodiments of the invention includes an elastic body of material having on one surface thereof a shape approximately complementary to posterior surfaces of a plurality of human vertebrae in a desired alignment, and having an another surface thereof a mounting structure capable of holding the article in a desired position adjacent a wall edge section. Numerous variations are possible. For example, the mounting structure may be an opening capable of forming a friction fit with the wall edge section. The article may be a single body of substantially homogeneous composition. The article may further include at least one arm forming one side of the opening of the mounting structure. The arms of an article including a mounting arm may include a spring urging the arm toward the opening. The shape of the article may be approximately complementary to posterior surfaces of a plurality of properly aligned human vertebrae includes a recess corresponding to at least one spinous process. The shape may also, or alternatively, be approximately complementary to posterior surfaces of a plurality of properly aligned human vertebrae includes a recess corresponding to at least one transverse process. The surface of the article may have a generally convex aspect on which recesses are defined, corresponding to processes of the plurality of human vertebrae.

According to other aspects of embodiments of the invention, there is provided a kit including plural articles according to the various aspects of embodiments of the invention described above, each complementary to different plural vertebrae. The kit may further include an extension block having a surface that can receive pressure applied by a person's hands on one surface, and another surface having a mounting structure capable of holding the article in a desired position adjacent a wall edge section.

According to yet other aspects of embodiments of the invention, a method aligning plural vertebrae in a human having need thereof, comprises: providing an elastic support having a surface shape complementary to a plurality of aligned vertebrae; relaxing spinal muscles adjacent the plural vertebrae to be aligned; and pressing the spine against the elastic support so as to align the spine. Variations on the method are, of course, also possible. For example, the elastic support may be removably provided on a jamb of a door. The pressing may be done against another jamb of the door. Relaxing, according to yet another variation may further comprise: resting the plural vertebrae against the elastic support; inhaling deeply; and plural vertebrae; exhaling slowly with the plural vertebrae maintained against the elastic support. The method may further include flexing the spine in a reverse kyphosis arc.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is an axial (from the bottom) view of a vertebra and associated ribs;

FIG. 2 is a sagittal (from the right side) view of a representative group of three thoracic vertebrae;

FIG. 3 is a perspective view of a massager useful in connection with aspects of embodiments of the invention;

FIG. 4 is a plan view of the massager of FIG. 3, with rollers closed;

FIG. 5 is a plan view of the massager of FIGS. 3 and 4 with rollers open;

FIG. 6 is an axial view of a vertebra showing the location of the paraspinous muscles;

FIG. 7 is an axial (from the bottom) view of the vertebra of FIG. 6, illustrating schematically the use of the massager of FIGS. 3, 4 and 5;

FIG. 8 is a perspective view of an aligner device according to and useful in connection with various aspects of embodiments of the invention;

FIG. 9 is an axial (from the bottom) view of a vertebra pressed into the aligner device of FIG. 8;

FIG. 10 is a sagittal (from the right side) cross-sectional view of a representative group of three thoracic vertebrae pressed into the aligner device of FIGS. 8 and 9;

FIG. 11 is a perspective view of a mounting block used in connection with either the massager of FIGS. 3, 4 and 5, or the aligner device of FIGS. 8 and 9;

FIG. 12 is a perspective view of a spring clamp mount used in connection with either the massager of FIGS. 3, 4 and 5, or the aligner device of FIGS. 8 and 9;

FIG. 13 is a perspective view of a cam mount used in connection with either the massager of FIGS. 3, 4 and 5, or the aligner device of FIGS. 8 and 9;

FIG. 14 is a perspective view of the massager of FIGS. 3, 4 and 5, mounted to a door jamb using the mounting block of FIG. 11;

FIG. 15 is a perspective view of the aligner device of FIGS. 8 and 9, mounted to a door jamb using the mounting block of FIG. 11;

FIG. 16 is a perspective view of the aligner device of FIGS. 8 and 9, mounted to a door jamb using the mounting block of FIG. 11 and further including a spacer usable with either the aligner device or the massager device; and

FIG. 17 is a perspective view of a spacer device mountable to a door jamb and usable separately from the massager device or the aligner device.

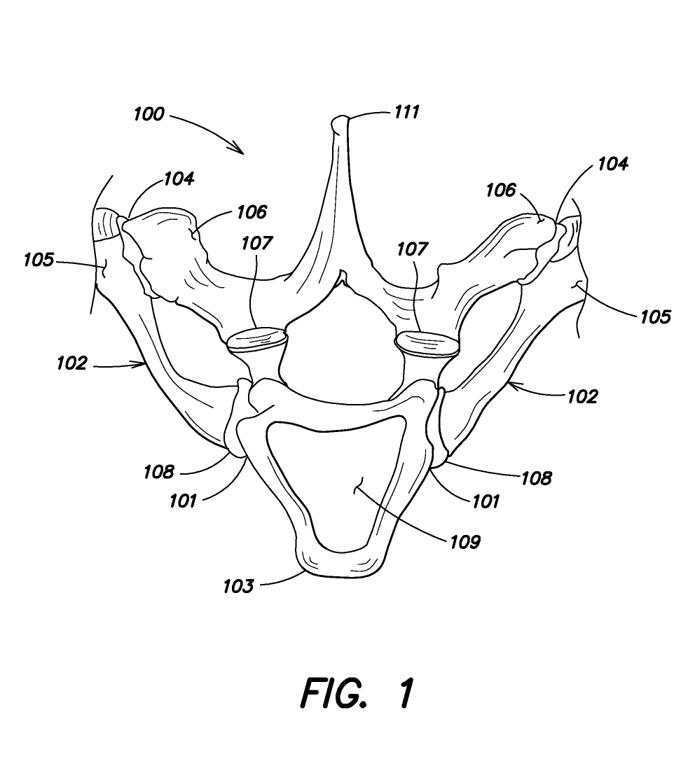

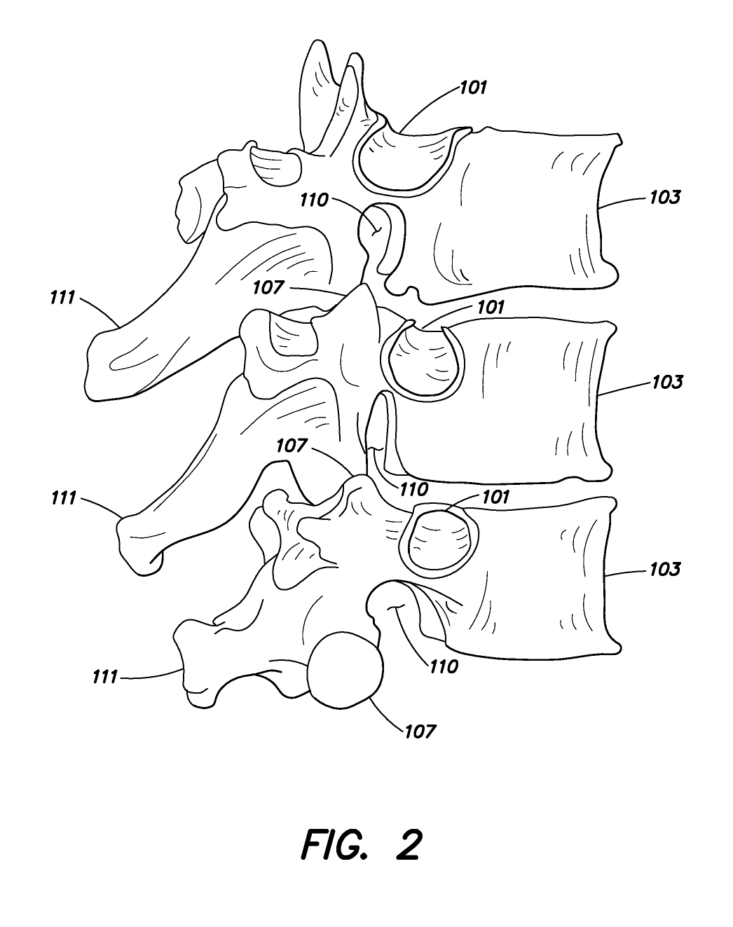

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

According to some aspects of embodiments of the invention, realignment of the vertebrae of a human spine is achieved by first relaxing the supporting musculature, for example through the use of massage, followed by pressing the bony prominences of that portion of the spine requiring realignment against a firm, but flexible, form promoting alignment. The degree of firmness or flexibility of the form promoting alignment may be selected by the designer to produce a desired degree of comfort while also providing sufficient resistance to pressure to realign vertebrae that are held out of alignment by tight or spastic muscles or related tissues.

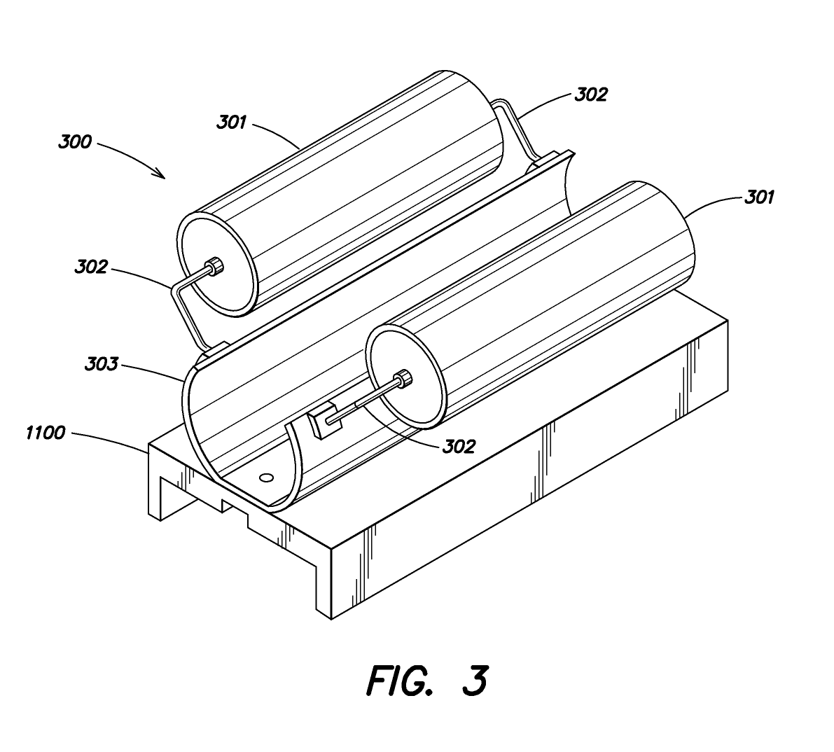

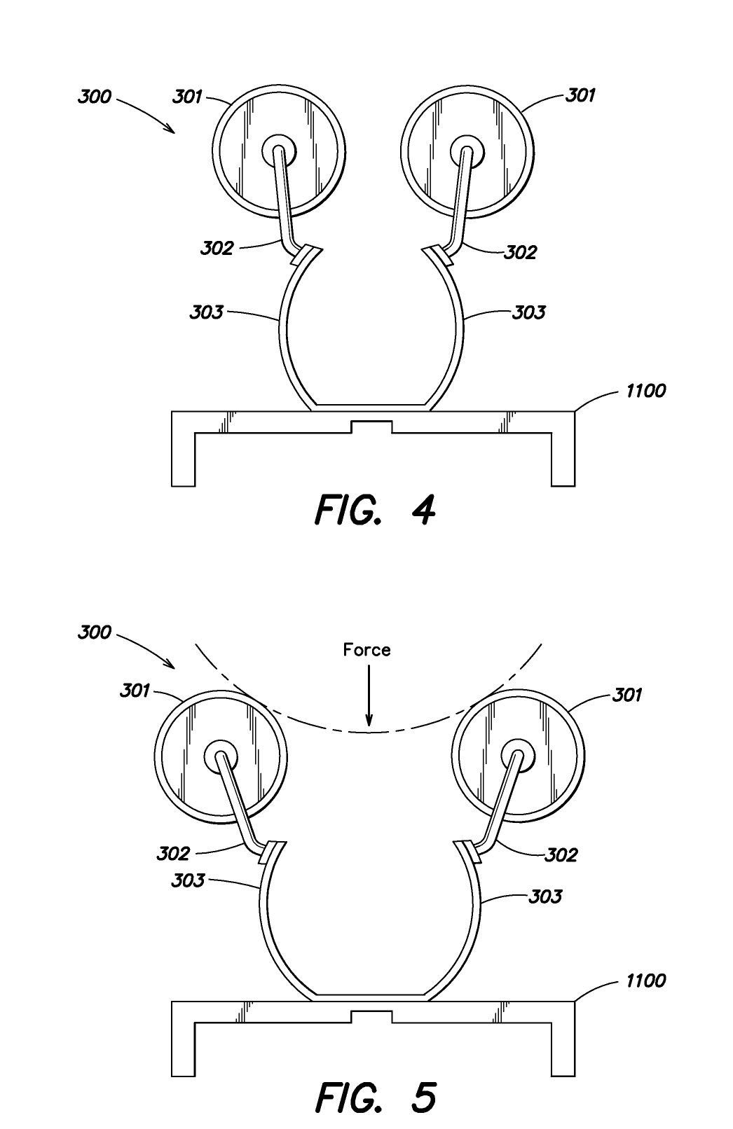

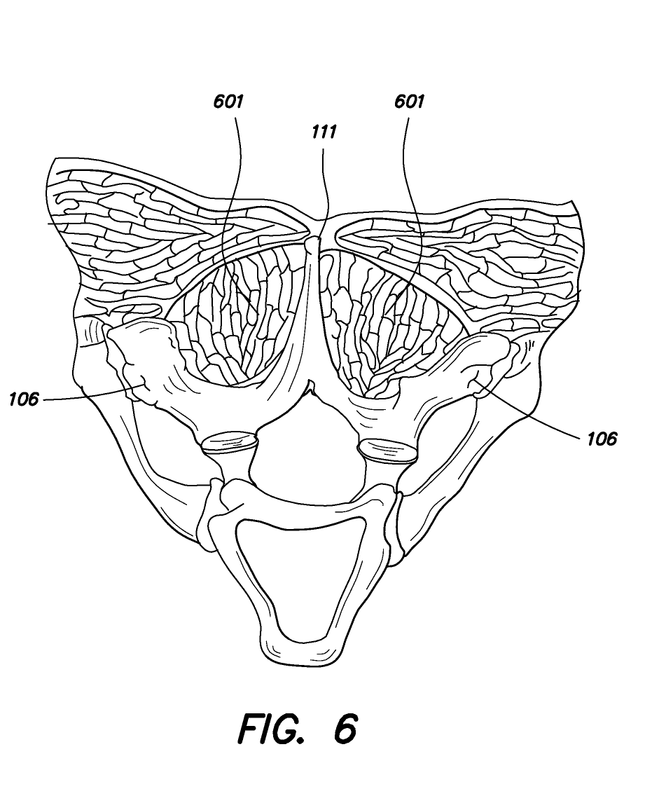

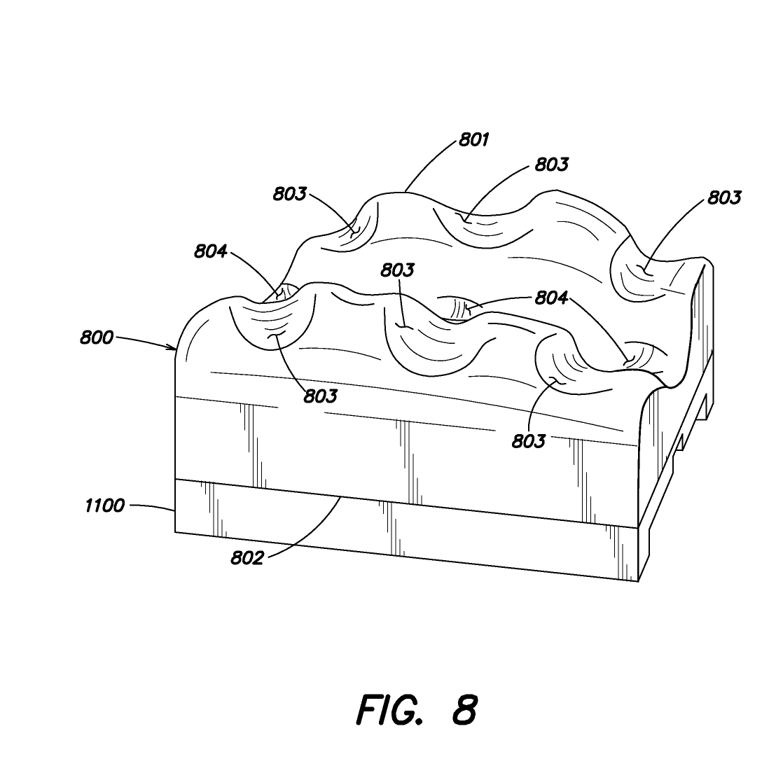

Various apparatus may be used in connection with the above method or other methods. For example, a mechanical massage device is described (See FIGS. 3, 4 and 5), which specifically relaxes the paraspinous muscles, which run vertically along the spinal column, perpendicular to their long axis (See FIG. 6), so as to allow alignment changes. Without such relaxation, the paraspinous muscles tend to hold the vertebrae in the current alignment or misalignment, despite manipulation, especially if the paraspinous muscles are in spasm. Another device useful in connection with the foregoing and other methods is a flexible, but supportive form, promoting the correct alignment of the vertebrae, which can be conveniently placed or mounted for the user to self-apply the required amount of pressure to achieve the desired realignment (See FIG. 8).

Both the massager and the alignment device that will now be described can be mounted in a door jamb, on a wall edge or by other suitable means, or can be placed on the floor to be used in a passive mode. The source of the required force or pressure can be an assistant, a person's own arms or the person's own weight. Any other suitable source of pressure can also be used.

The massager device is now described in connection with FIGS. 3, 4 and 5.

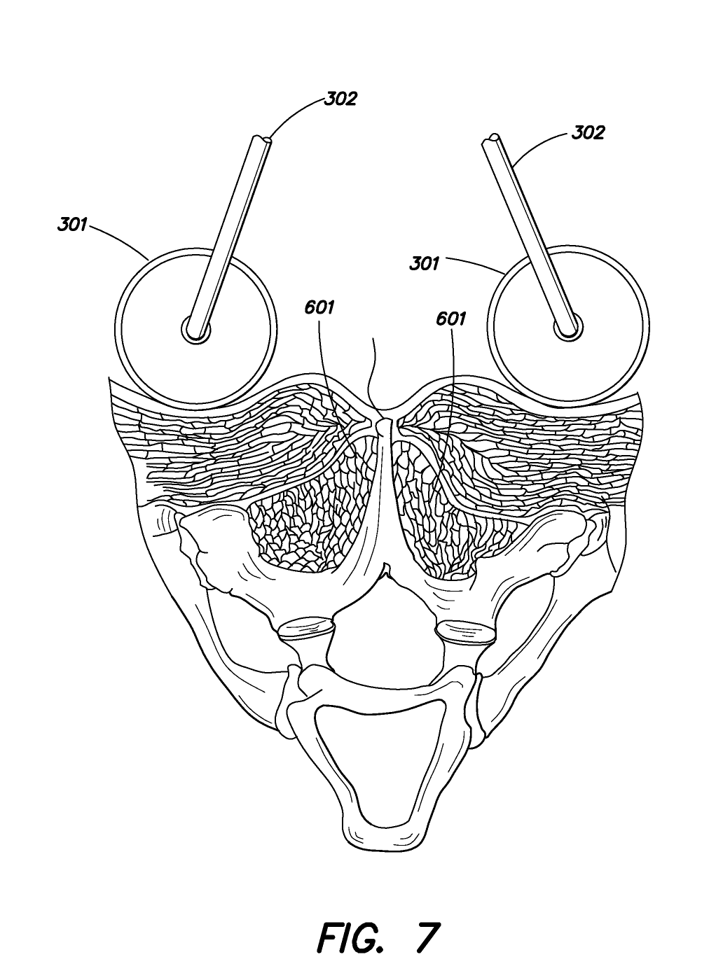

The massager 300 can include two or more rollers 301, attached by arms 302 to a central spring 303, as shown in FIG. 3. The spring 303 biases the arms 302 carrying the rollers 301 together, so that a force applied between the rollers 301 is required to separate them. Each of the rollers 301, can include multiple segments, all of which are carried on common, optionally flexible, axles attached to the arms 302. As shown in FIG. 6, the paraspinous muscles 601 lie in the space between the transverse processes 106 and the spinous process 111. These muscles 601 extend vertically along the length of the spine. As shown in FIG. 7, when a person leans the affected portion of their spine against the rollers 301 of the massager 300, the shape of the spine presses the rollers 301 apart causing them to massage the paraspinous muscles 601 perpendicular to their long axis by flattening them against the ribs 102 and transverse processes 106. The paraspinous muscles 601 are pushed laterally away from the midline, perpendicular to their long axis.

In addition to or alternatively to using the massager device 300, other suitable muscle relaxing techniques can be used. For example, whether used with or without the muscle relaxing device, deep breathing exercises can be used.

After performing the muscle relaxation, a realignment method is performed. The person rests or presses the affected portion of the spine against a firm, but flexible, alignment form. The alignment form has a plurality of recesses, approximately complementary to posterior surfaces of a plurality of properly aligned vertebrae. Thus, when the person applies sufficient pressure, the vertebrae are forced into approximate alignment, which position they will tend to maintain for a period of time after performing the realignment method.

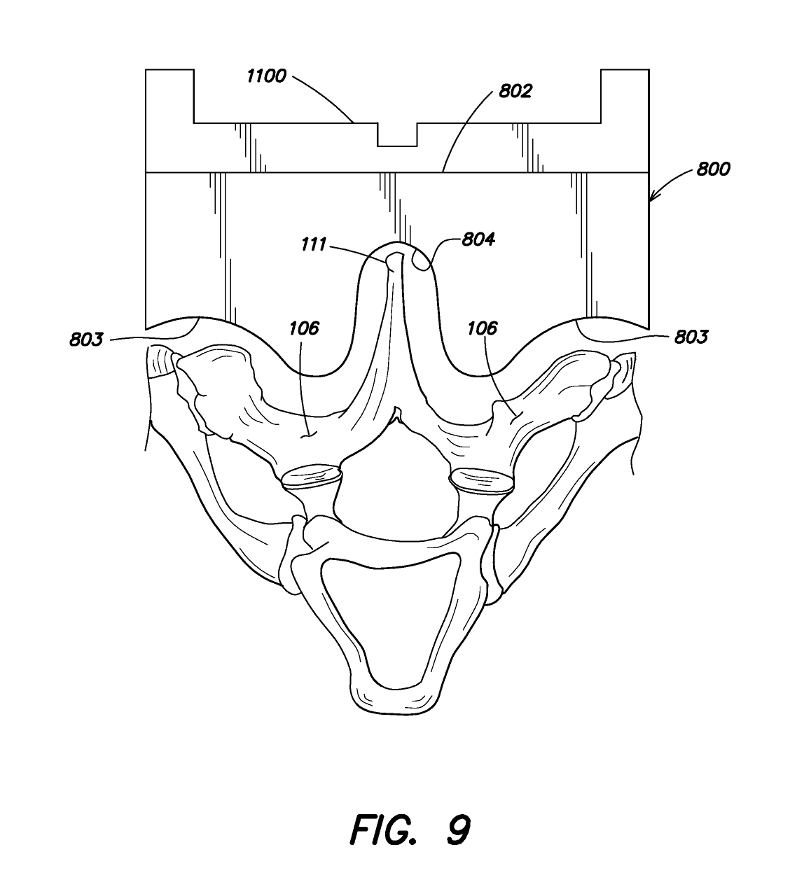

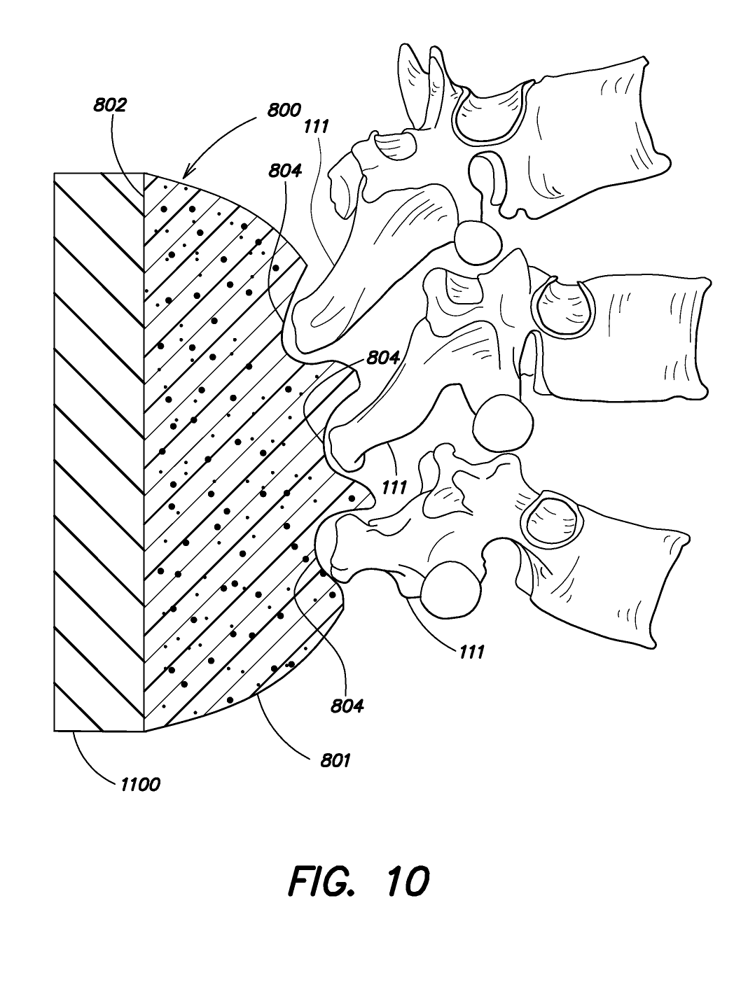

An exemplary alignment device embodying aspects of the invention is first described in connection with FIGS. 8, 9 and 10. Then a method of use of the alignment device, also embodying aspects of the invention is described, also in connection with FIGS. 8, 9 and 10.

The exemplary alignment device 800 is a body of firm, but flexible, material having a front side 801 and a back side 802. The back side is adapted for the type of mounting the alignment device will be put to, described below, while the front side is adapted to align the vertebrae as now described. The material of which the alignment device is constructed may be any suitable material firm enough to hold its general shape during use as described herein, but flexible enough to be comfortable during use as described herein. Various foams, gels and elastomers are known which are suitable. The body of the alignment device has recesses which comfortably receive properly aligned vertebrae without creating pressure points that could cause pain or discomfort.

The front side 801 of the device 800 has a generally curved shape. The curve is not the natural curve of the spinal column, but rather a slightly exaggerated curve that produces a slight reverse kyphosis, or lordosis, of the spinal column in the region where applied as described below. Formed in reverse relief in the front side 801 surface of the device 800 are recesses 803, 804, corresponding to the spinal processes 106, 111, which protrude slightly through the skin and muscle tissue of the back.

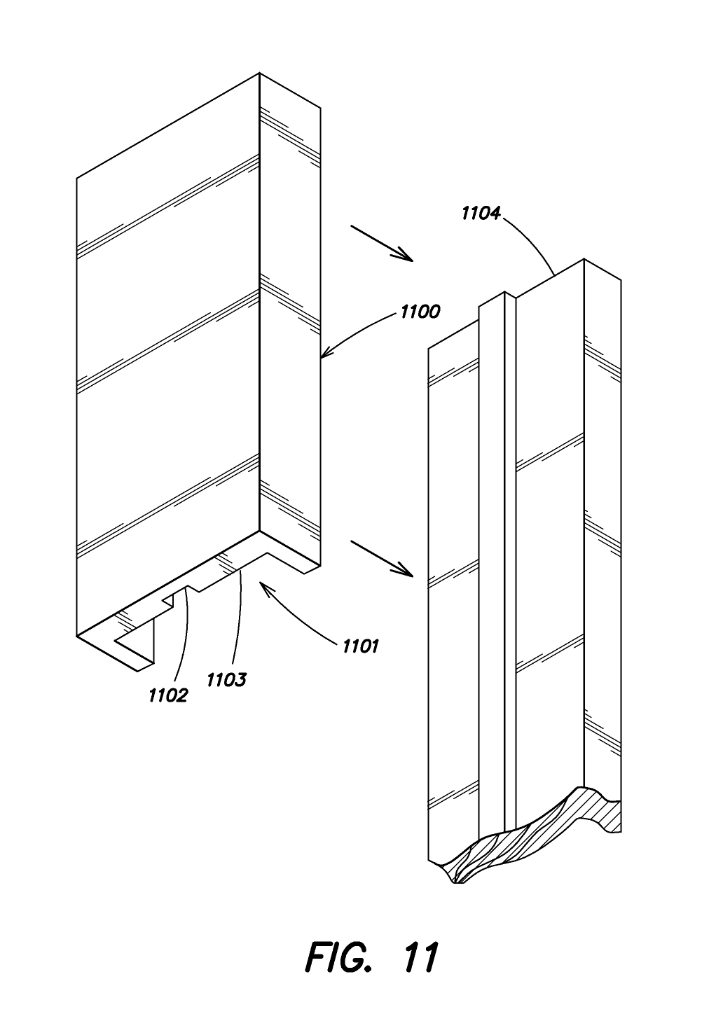

The back side 802 of the device 800 may include a mount, for example to hang the device 800 at a suitable height on a wall or on a door jamb. Particularly useful is mounting the device 800 on a door jamb, so three exemplary mounts are now described in connection with FIGS. 11, 12 and 13.

The mount 1100 of FIG. 11 includes a stepped channel 1101 which may be slipped onto a wall section or doorjamb 1104. The stepped channel 1101 includes gaps 1102, 1103, of varying sizes to facilitate a friction fit between the mount 1100 and the wall section or doorjamb 1104. Because the mount 1100 is constructed preferably as a unitary component with the aligner, e.g. FIG. 8, 800, it is of the same firm, but flexible, material which will form a tight friction fit with the wall section or door jamb 1104. If the combined weight of the aligner device 800 and mount 1100 is kept low, such a friction fit will be sufficient to suspend the aligner device 800 in a suitable position for use until the user can press their spine against the aligner device 800.

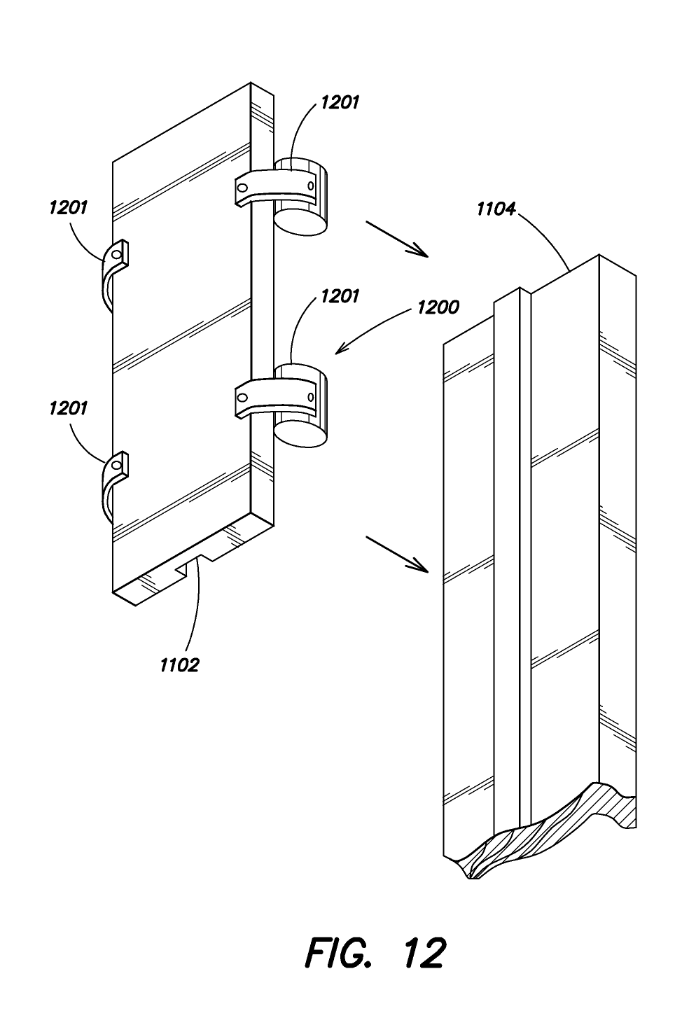

As shown in FIG. 12, a spring mount 1200 can be used in place of the mount 1100 shown in FIG. 11. The spring mount 1200 includes arms 1201 that clamp the wall section or door jamb 1104 with sufficient force to maintain the aligner device 800 and the mount 1200 in a suitable position for use until the user can press their spine against the aligner device 800.

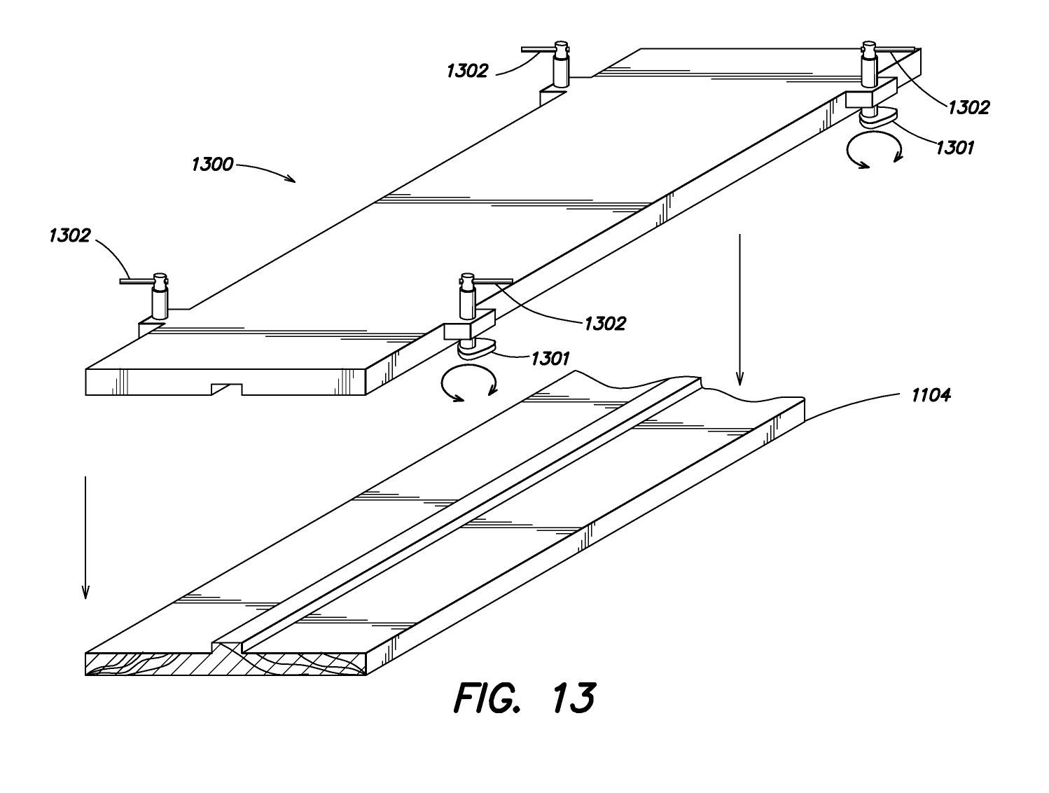

As shown in FIG. 13, a cam mount 1300 can alternatively be used in place of the mount 1100 shown in FIG. 11. The cam mount 1300 includes cams 1301 that when operated by levers 1302 clamp the wall section or door jamb 1104 with sufficient force to maintain the aligner device 800 and the mount 1200 in a suitable position for use until the user can press their spine against the aligner device 800.

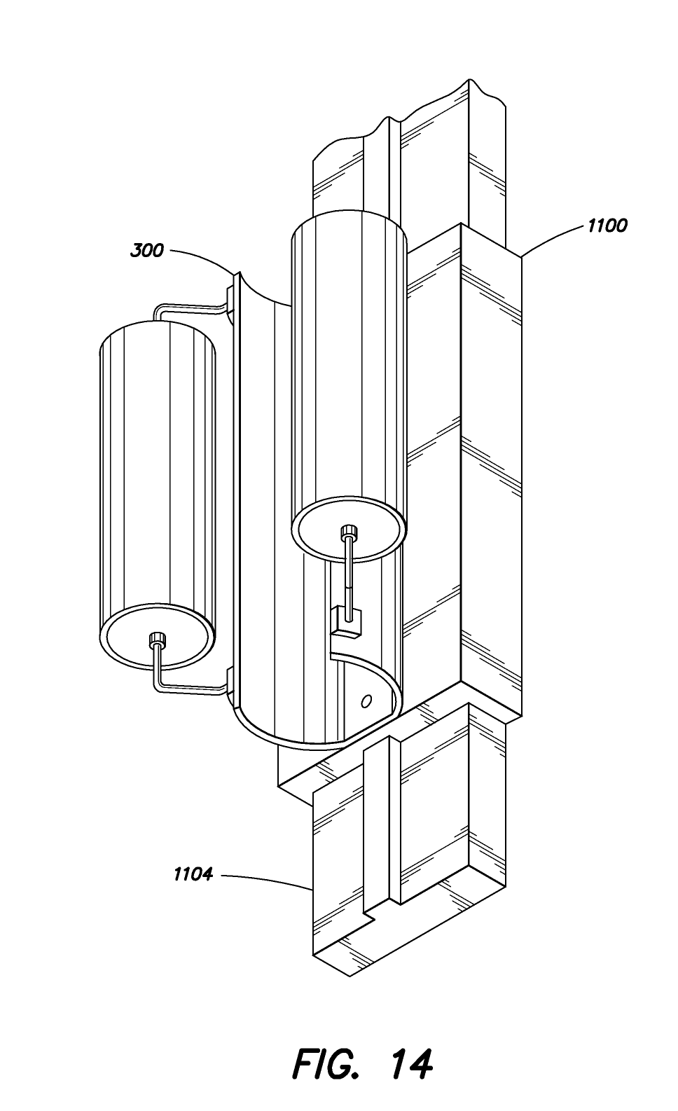

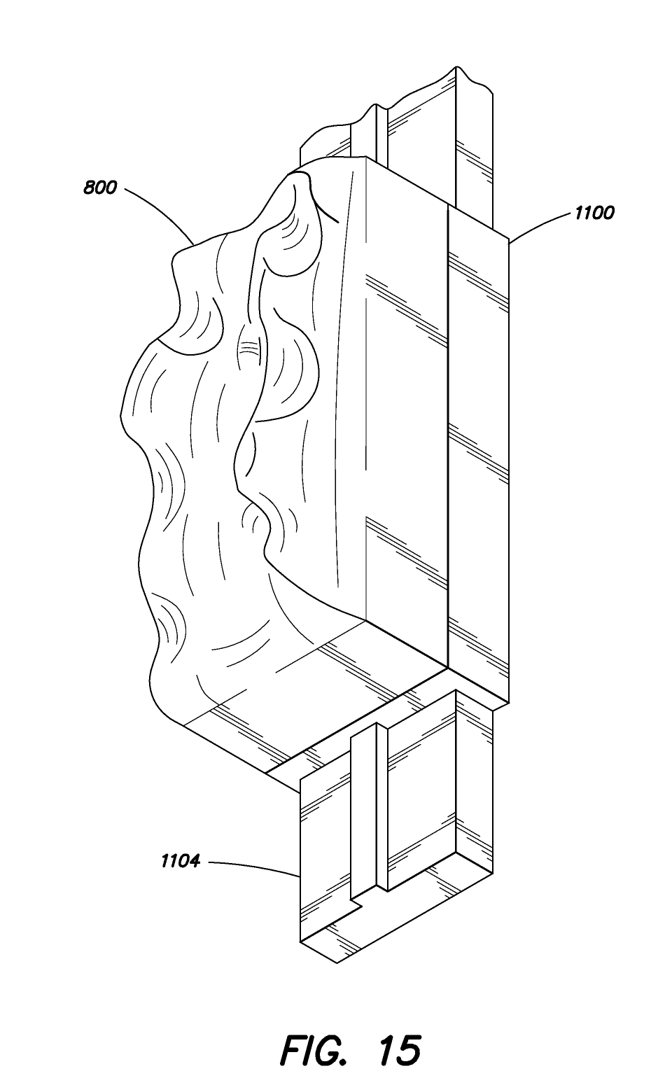

An application of the mount 1100 of FIG. 11 to suspend the massager of FIGS. 3, 4 and 5 from a door jamb is shown in FIG. 14. A similar application of the mount 1100 of FIG. 11 to suspend the aligner device of FIGS. 8, 9 and 10 from a door jamb is shown in FIG. 15. In each of the applications shown in FIGS. 14 and 15, any of the mounts described above, or any other suitable mount may be substituted.

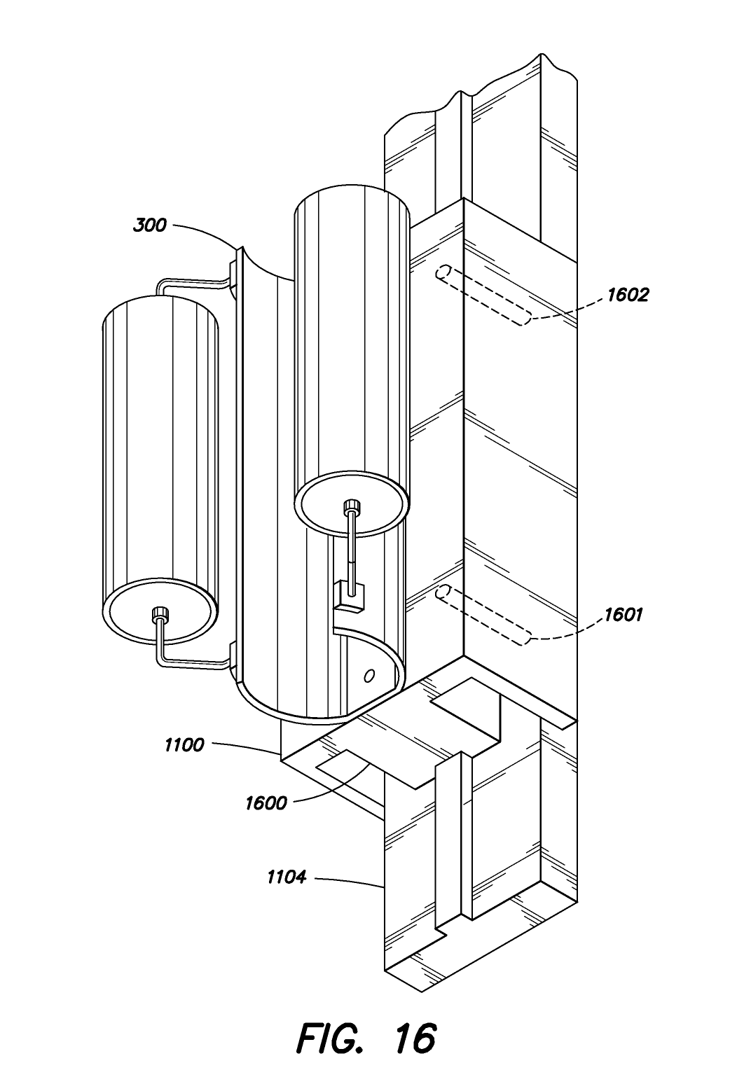

As shown in FIG. 16 either the massager or the aligner may be fitted with a spacer 1600 to allow an individual whose arms are too short to reach across a door opening to use the devices suspended from a door jamb. The spacer 1600 simply moves the massager or the aligner device away from the edge of the doorjamb 1104. In order to stabilize the massager or the aligner device in the extended position, the spacer 1600 may include one or more braces 1601, 1602.

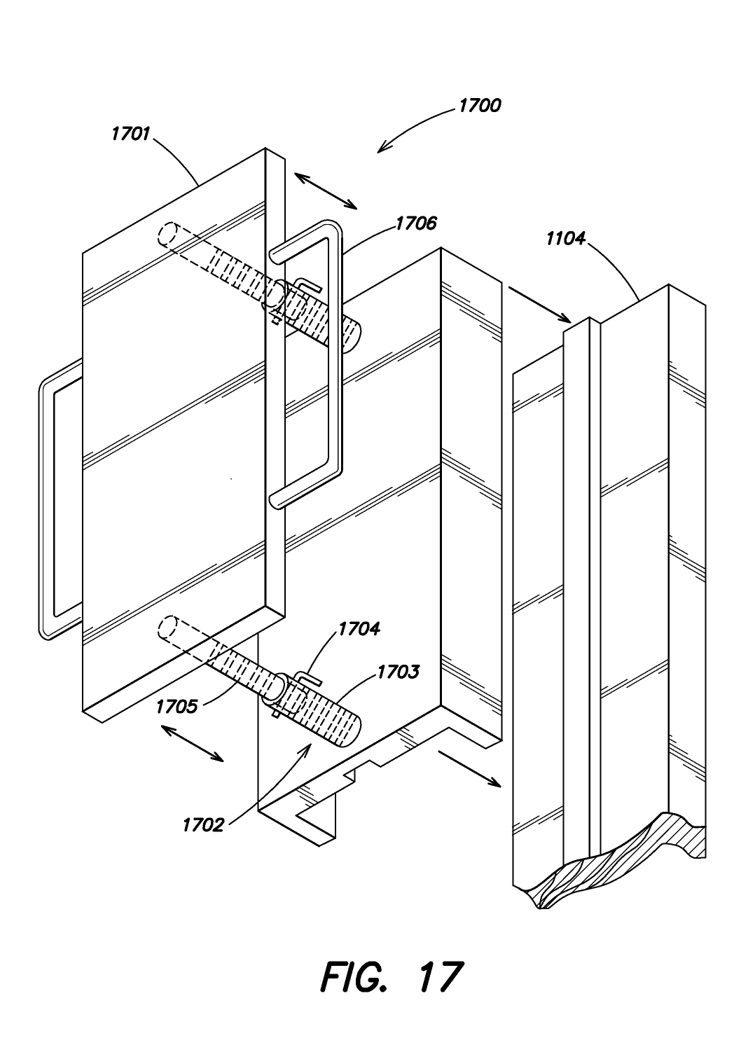

Alternatively to the spacer of FIG. 16, a separate, stand-alone spacer 1700 may be used, as shown in FIG. 17. The exemplary stand-alone spacer 1700 includes a mounting structure 1701, for example that of FIG. 11, 1100. A variable-spacing feature may be implemented, for example by a scissor-extension mechanism 1702. The stand-alone spacer 1700 may also include braces 1703, 1704 to stabilize it in the extended position. The spacer 1700 may further include an optionally padded surface or handles 1705 for the user to press against, so as to provide the requisite pressure between the user's back and the aligner device suspended from an opposite door jamb (not shown).

An exemplary method of improving the alignment of a person's vertebrae is now described.

The person first massages the paraspinous muscles to loosen them and relieve as much spasming of those muscles as possible. The massage may be provided using the massager device described above, or any other suitable means, for example an assistant massaging the muscles manually. After the paraspinous muscles have been loosened sufficiently, the person may proceed to the alignment portion of the method.

An alignment device, such as described above, is selected, for example from a kit of such devices matching different portions of a person's spinal column. The device is removably mounted at a suitable height on a door jamb or wall section where the person can reach to and press against an opposing door jamb or wall section. The person then relaxes their spine against the alignment device, with the vertebrae loosely aligned and fitted into the recesses of the alignment device. Finally, the person presses against the opposing door jamb or wall section, stretching the spinal column gently over the alignment device, producing the characteristic “crack.” The person then relaxes the pressure, the re-alignment having been accomplished.

Instead of using the aligner device in a doorway or archway with opposing door jambs or wall sections, a flat-backed aligner device can be laid on the floor, with the using lying on their back on top thereof. The requisite pressure may be provided by the person's own weight, or other suitable means, when practicing this method.

Another option includes using hook and loop tape fasteners to mount the aligner device or the massager device to a car seat or other seating apparatus to stretch against or over during extended periods of sitting. Yet another option includes using suction cups to attach the aligner device or the massager to a counter edge, table edge, tile wall, shower stall wall (one which is a fixed part of the building structure, not a glass enclosure wall), or the like.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An article comprising an elastic body of material having on one surface thereof a shape approximately complementary to posterior surfaces of a plurality of human vertebrae in a desired alignment, and having an another surface thereof a mounting structure capable of holding the article in a desired position.

2. The article of claim 1, wherein the mounting structure is an opening capable of forming a friction fit with a wall edge section.

3. The article of claim 2, wherein the article is a single body of substantially homogeneous composition.

4. The article of claim 2, wherein the article further comprises at least one arm forming one side of the opening of the mounting structure.

5. The article of claim 4, wherein the arms includes a spring urging the arm toward the opening.

6. The article of claim 1, wherein the shape approximately complementary to posterior surfaces of a plurality of properly aligned human vertebrae includes a recess corresponding to at least one spinous process.

7. The article of claim 1, wherein the shape approximately complementary to posterior surfaces of a plurality of properly aligned human vertebrae includes a recess corresponding to at least one transverse process.

8. The article of claim 1, wherein the one surface thereof has a generally convex aspect on which recesses are defined, corresponding to processes of the plurality of human vertebrae.

9. A kit including plural articles according to claim 1, each complementary to different plural vertebrae.

10. A kit including an article according to claim 1, and an extension block having a surface that can receive pressure applied by a person's hands on one surface, and another surface having a mounting structure capable of holding the article in a desired position adjacent a wall edge section.

11. A method aligning plural vertebrae in a human having need thereof, comprising:
providing an elastic support having a surface shape complementary to a plurality of aligned vertebrae;
relaxing spinal muscles adjacent the plural vertebrae to be aligned; and
pressing the spine against the elastic support so as to align the spine.

12. The method of claim 11, wherein the elastic support is removably provided on a jamb of a door.

13. The method of claim 12, wherein the pressing is done against another jamb of the door.

14. The method of claim 11, wherein relaxing further comprises:
resting the plural vertebrae against the elastic support;
inhaling deeply; and plural vertebrae;
exhaling slowly with the plural vertebrae maintained against the elastic support.

15. The method of claim 11, further comprising flexing the spine in a reverse kyphosis (lordosis) arc.
